(19) United States
(12) Patent Application Publication
(10) Pub. No.: US 2008/0183115 A1
(43) Pub. Date: Jul. 31, 2008

(54) CAST AIR DELIVERY SYSTEMS, CAST VENTILATION SLEEVES AND METHODS OF MANUFACTURING CAST SLEEVES

(75) Inventor: David Pierce, Renton, WA (US)

Correspondence Address: PERKINS COIE LLP;PATENT-SEA, P.O. BOX 1247, SEATTLE, WA, 98111-1247, US

(73) Assignee: Global Concepts IP, LLC., Renton, WA (US)

(21) Appl. No.: 11/866,384

(22) Filed: Oct. 2, 2007

Related U.S. Application Data
(60) Provisional application No. 60/849,159, filed on Oct. 2, 2006.

(51) Int. Cl. A61F 5/00

(52) U.S. Cl. 602/13; 602/5

(57) ABSTRACT

Cast air delivery systems, cast ventilation sleeves and methods of manufacturing cast sleeves are disclosed herein. In one embodiment, a method of manufacturing cast ventilation sleeves includes forming an elongated cylindrical sleeve from a breathable, elastomeric material. The method can further include providing an air delivery tube configured to receive pressurized air. The method can also include disposing a plurality of spaced apart holes in the air delivery tube, wherein the holes can be positioned to disperse the pressurized air along a tube length. Additionally the method can include coupling the air delivery tube to the elongated sleeve to form a cast ventilation sleeve stock. The air delivery tube can be positioned to be generally parallel to a sleeve length and the tube length can be generally equal to a sleeve length. The elongated sleeve can be cut to a selectable length for use under an orthopedic cast.

### CROSS-REFERENCE TO RELATED APPLICATION

This application claims priority to U.S. Provisional Patent Application No. 60/849,159, filed Oct. 2, 2006, entitled “IMPROVEMENT TO CAST VENTILATION SLEEVE,” and which is incorporated by reference herein.

### TECHNICAL FIELD

The present disclosure is directed to systems and methods for venting orthopedic casts, and to methods of manufacturing cast sleeves for orthopedic cast ventilation.

### BACKGROUND

Orthopedic casts are commonly used in the medical field for immobilization of broken or fractured bones, such as arms, legs, etc. Following bone resetting and/or other surgical procedures, a medical technician must spend time to correctly apply and sculpt a rigid cast to aptly support and provide protection for the affected broken or fractured bone. Typically, the cast is worn for a period of time necessary to allow the bone to heal, such as weeks or months. During this length of time, the cast is permanently affixed to the person.

Conventional casts can be formed from plaster or other resin-impregnated bandage material (e.g., fiberglass) that hardens after becoming wet. Stockinettes, or other wrapped fabric is used between the rigid cast and the skin of the patient to provide protection against skin abrasion and resulting bacterial infections during the healing period. Regardless of the use of the stockinettes, patients wearing conventional casts commonly endure skin irritation and itching due to trapped moisture and bacterial accumulation. Furthermore, patients must also deal with accumulation of mal odors resulting from the bacterial build-up between the cast and the skin.

Some aeration devices have been used to supply air for drying and/or reducing skin irritation beneath the cast; however, these devices are expensive, require additional layers of material and are time consuming for the medical technician to correctly incorporate into the cast system.

### SUMMARY

The following disclosure describes several embodiments of cast air delivery systems and cast ventilation sleeves for air delivery and dispersion beneath orthopedic casts. One aspect of the disclosure is directed to integrated air delivery systems having cast ventilation sleeves and methods of manufacturing cast ventilation sleeves for use with orthopedic casts to provide a comfortable and easy-to-apply air delivery method to relieve patient discomfort and decrease mal odors due to bacterial overgrowth, perspiration, etc.

In one embodiment, a method of manufacturing cast ventilation sleeves includes forming an elongated cylindrical sleeve having a sleeve length with a first sleeve end and a second sleeve end opposite from the first sleeve end and having a first major cross-sectional dimension (e.g., diameter). The elastomeric material can be configured to expand to a second major cross-sectional dimension greater than the first major cross-sectional dimension. The method can further include providing an air delivery tube configured to receive pressurized air. The air delivery tube can have a tube length with a first tube end and a second tube end opposite from the first tube end. The method can also include disposing a plurality of spaced apart holes in the air delivery tube, wherein the holes can be positioned to disperse the pressurized air along the tube length. Additionally the method can include coupling the air delivery tube to the elongated sleeve to form a cast ventilation sleeve stock. The air delivery tube can be positioned to be generally parallel to the sleeve length such that the first tube end is oriented toward the first sleeve end and the second tube end is oriented toward the second sleeve end.

In another embodiment, a method for manufacturing stockinettes for ventilating orthopedic casts is provided. The method can include providing an air delivery tube having a first open end and a second open end. After providing the air delivery tube, the method can include perforating at least a portion of the air delivery tube to provide spaced apart holes between the first and second open ends. The method can further include forming a flexible tubular sleeve having a sleeve length and formed from a material suitable to be worn next to a patient's skin. The method can also include attaching the perforated air delivery tube to the flexible tubular sleeve such that the air delivery tube is positioned to align with the sleeve length.

In a further embodiment of the disclosure, a stock of flexible material for use as a liner between a patient's skin and an orthopedic cast is provided. The stock can include an elongated sleeve of breathable material having a sleeve length and having inner and outer sleeve surfaces. The stock can also include an air delivery tube coupled to the elongated sleeve in an orientation generally parallel to the sleeve length. The air delivery tube includes a plurality of holes spaced apart along a tube length. The air delivery tube is configured to receive pressurized air and disperse the air through the holes.

Another embodiment of the disclosure is directed toward a cast ventilation system. The cast ventilation system can include a cast ventilation sleeve configured to be worn by the patient beneath an orthopedic cast and an air supply. The cast ventilation sleeve can include an elastomeric material configured to be worn next to a patient's skin, wherein the elastomeric material surrounds and expands to cover a patient body part. The sleeve also includes an air delivery tube coupled to the elastomeric material, the air delivery tube having a plurality of spaced apart air dispersing holes. The air dispersing holes can be positioned to deliver air adjacent to the patient's skin and beneath the orthopedic cast. The cast ventilation sleeve included in the system can be formed from a manufacturing process that includes (a) forming an elongated cylindrical sleeve from the elastomeric material, the elongated sleeve having a first length, (b) coupling the air delivery tube to the elongated sleeve such that the air delivery tube extends over the first length, and (c) cutting the elongated sleeve and the air delivery tube in a direction generally perpendicular to the first length to form the cast ventilation sleeve having a second length less than the first length. The second length can be selected to accommodate an orthopedic cast length. The air supply can be releaseably connected to the air delivery tube for providing pressurized air to the air delivery tube.

### BRIEF DESCRIPTION OF THE DRAWINGS

In order that advantages of the disclosure will be readily understood, a more particular description of aspects of the disclosure briefly described above will be rendered by reference to specific embodiments and the appended drawings. Understanding that these drawings depict only typical embodiments of the disclosure and are not therefore to be considered to be limiting of its scope, the disclosure will be described and explained with additional specificity and detail through the use of the accompanying drawings.

FIG. 1 is a schematic cut-away view of an orthopedic cast with an integrated air delivery system in accordance with an embodiment of the disclosure.

FIG. 2 is an isometric view illustration of a cast ventilation sleeve with integrated air delivery tubes, and cut to size for application to a patient's limb in accordance with an embodiment of the disclosure.

FIG. 3A illustrates a portion of the cast ventilation sleeve (indicated in FIG. 1) with the air delivery tube attached to an exterior surface of a stockinette material and in accordance with an embodiment of the disclosure.

FIG. 3B illustrates a portion of a cast ventilation sleeve with an air delivery tube positioned inside a sleeve receiving channel and in accordance with an embodiment of the disclosure.

FIG. 4 is a perspective view of an embodiment of an air supply nozzle for delivering air into an air delivery tube in accordance with an embodiment of the disclosure.

FIG. 5 is a perspective view of an air delivery tube closure plug in accordance with an embodiment of the disclosure.

FIG. 6 is schematic top perspective view illustration of a cast ventilation sleeve configured to receive air from an air supply in accordance with an embodiment of the disclosure.

FIG. 7 is schematic top perspective view illustration of a cast ventilation sleeve configured to receive air from a portable compressed can of air in accordance with an embodiment of the disclosure.

FIG. 8 is a perspective view illustration of a roll of cast ventilation sleeve stock in accordance with an embodiment of the disclosure.

FIG. 9 is a flow chart illustrating a method of manufacturing cast ventilation sleeves in accordance with an embodiment of the disclosure.

FIG. 10 is a flow chart illustrating a method of manufacturing stockinettes for ventilating orthopedic casts in accordance with an embodiment of the disclosure.

### DETAILED DESCRIPTION

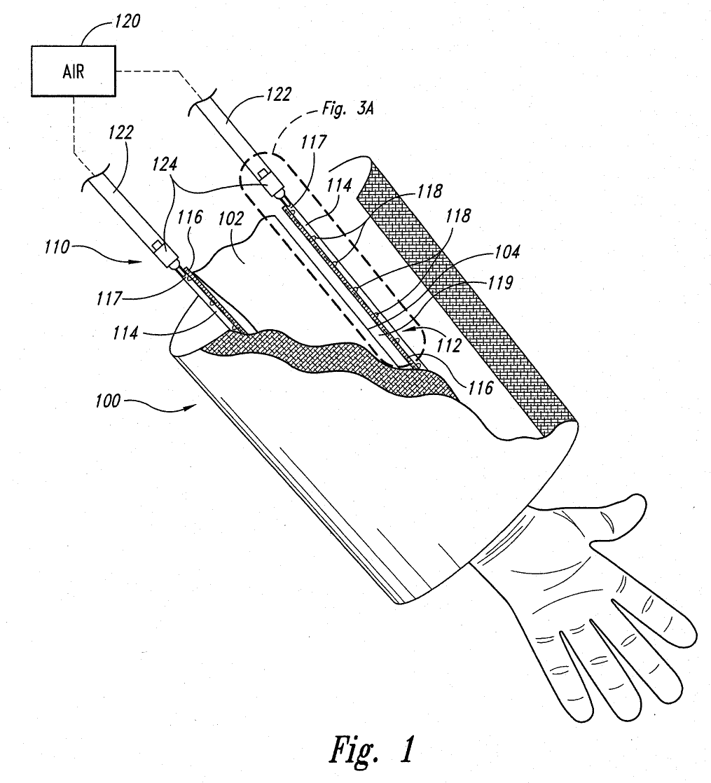

FIG. 1 is a schematic cut-away view of an orthopedic cast 100 with an integrated air delivery system 110 in accordance with an embodiment of the disclosure. The orthopedic cast 100 can be a rigid cast formed of plaster or other resin-impregnated material that, after becoming wet, can be sculpted around a body part, such as an arm, leg, torso, etc., and dried to form a hardened protective layer for the affected body part. As illustrated schematically in FIG. 1, the orthopedic cast 100 is applied to a patient's forearm 102, such as a forearm 102 with a broken bone for the purpose of immobilizing and protecting the healing bone. One of ordinary skill in the art will recognize a variety of materials and methods used, e.g., by medical practitioners, to form protective orthopedic casts, such as the cast 100.

The integrated air delivery system 110 is intermediate the orthopedic cast 100 and a patient's skin 104. As illustrated, the integrated air delivery system 110 includes a cast ventilation sleeve 112 having one or more air delivery tubes 114 coupled to the sleeve. The cast ventilation sleeve 112 can be formed of an elastomeric material or other stretchable material suitable for expanding to encase and conform to an afflicted body part. For example, in one embodiment, the cast ventilation sleeve 112 is formed from a breathable stockinette material 116, such as cotton, nylon, or other breathable fabric suitable for wearing against the patient's skin 104.

As illustrated in FIG. 1, the system 110 includes the one or more air delivery tubes 114 coupled to the stockinette material 116. The individual air delivery tubes 114 are configured to receive pressurized air from an air source 120 through a first tube end 117. In one embodiment, the air delivery tubes 114 can be constructed from a flexible synthetic material, such as a polyethylene polymer material, or other synthetic polymer material suitable for receiving pressurized air therein. In some embodiments, the air delivery tube 114 can be formed of a thin, flexible synthetic material such that the tube 114 will rest in a collapsed configuration when not receiving air and have substantially the thickness of the synthetic material. Following air delivery, the tube 114 can be configured to inflate and allow the air to be dispersed substantially equally along the tubes 114. In other embodiments, the air delivery tubes 114 can be formed from a variety of thermoplastics (e.g., polystyrene, polyethylene, PVC, etc.) molded to form an air delivery tube. In yet a further embodiment, the air delivery tubes 114 can be formed from densely woven fabric or nylon material suitable to be at least partially impermeable to pressurized air administered within the tube.

The air source 120 can deliver the pressurized air to the air delivery tubes 114 via one or more air supply lines 122. The air supply lines 122 can releaseably engage the first tube end 117 with an air delivery nozzle 124 when aeration beneath the orthopedic cast 100 is desired. In another embodiment, the air delivery nozzle 124 can be permanently or semi-permanently affixed to the air delivery tubes 114 such that the air supply line 122 can releaseably engage the air delivery nozzle 124. Each air delivery tube 114 is provided with a plurality of holes 118 spaced apart from one another and positioned along the air delivery tube 114 to disperse the administered pressurized air to a space 119 between the patient's skin 104 and the orthopedic cast 100.

In the embodiment illustrated in FIG. 1, the air delivery tube 114 can be coupled to the stockinette material 116 such that the stockinette material is between the air delivery tube 114 and the patient's skin 104. In this arrangement, air flow from the holes 118 can be directed toward and through the stockinette material 116 before reaching the patient's skin 104. As described above, the stockinette material 116 can be an elastomeric material configured to stretch across the skin 104. As the stockinette material 116 stretches, thereby opening the mesh of the material, air flow from the holes 118 can pass easily through the material 116 to dry the skin 104. Furthermore, the stretched stockinette material 116 can also assist in air dispersal laterally along the outer surface of the skin 104 and along the cast ventilation sleeve 112 to facilitate drying of the sleeve. In one arrangement, air flow from the holes 118 does not directly impact the skin region directly below the holes, but the air is spread by the stretched stockinette material 116 to distal regions of the skin as well. Accordingly, the holes 118 perforating the air delivery tube 114 can provide sufficient air flow throughout the entire space 119 between the between the patient's skin 104 and the orthopedic cast 100 regardless of the position of the air delivery tube 114. In another embodiment, however, the air delivery tube 114 may be integrated within the stockinette material 116, or in a further embodiment, the air delivery tube may be between the skin 104 and the stockinette material.

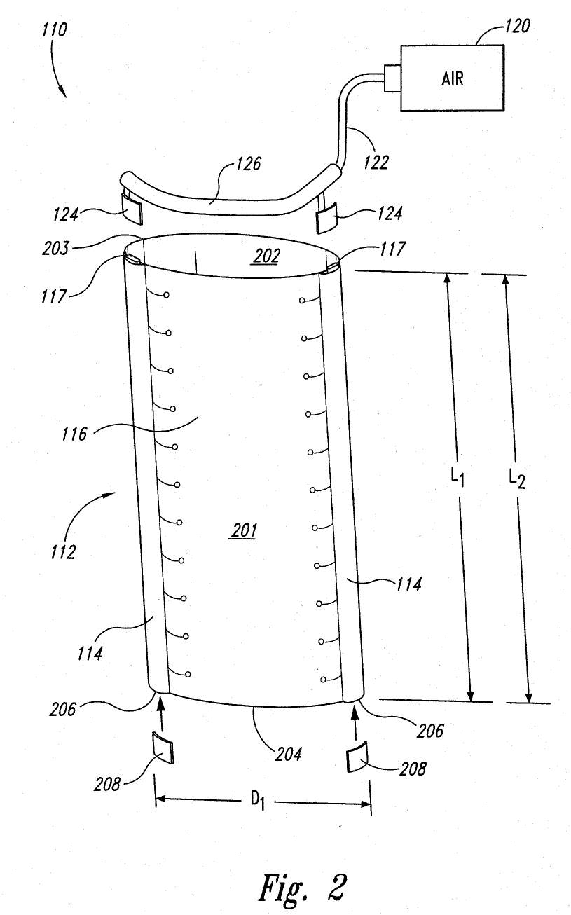

FIG. 2 is a top perspective view of a cast ventilation sleeve 112 with air delivery tubes 114 in accordance with an embodiment of the disclosure. As illustrated, the cast ventilation sleeve 112 can be substantially tubular having an exterior surface 201 for facing toward the cast 100 and an interior surface 202 facing toward the patient's skin 104 (shown in FIG. 1). The cast ventilation sleeve 112 is provided with a major cylindrical cross-sectional dimension D₁ (e.g., a diameter of approximately 3-6 inches) and has a sleeve length L₁ with a first sleeve end 203 and a second sleeve end 204 opposite the first sleeve end 203. The sleeve length L1 can be selected or be an otherwise suitable length for use beneath orthopedic casts (e.g., 2-3 feet). The first and second sleeve ends 203 and 204 can be open ends such that the cast ventilation sleeve 112 can be transitioned over a hand or foot, for example, to an upper arm or leg portion respectively. In one embodiment, the cast ventilation sleeve 112 can accommodate a body part, such as an arm or leg, by expanding to a second cross-sectional dimension (not shown) that is greater than the first cross-sectional dimension D₁.

The air delivery tubes 114 have a tube length L₂ between the first tube end 117 and a second tube end 206 opposite the first tube end 117. The tube length L₂ can be substantially aligned with the sleeve 112 in a generally parallel orientation such that the tubes 114 extend along the sleeve length L₁. In some embodiments, the tube length L₂ can be approximately equal to the sleeve length L₁. In some embodiments, the cast ventilation sleeve 112 may have a single air delivery tube 114 coupled along the sleeve length L₁; however, it will be appreciated by one of ordinary skill in the art that a plurality of air delivery tubes 114, such as the two tubes 114 shown in FIG. 2, may be aligned and coupled to the sleeve 112. In general, the air delivery tubes 114 can be spaced apart from other ones of the air delivery tubes. In one embodiment, the air delivery tubes 114 are moveable with a portion of the cast ventilation sleeve material, such as the elastomeric material such that the air delivery tubes 114 can be positioned over selectable portions of a patient's encased body part when the sleeve 112 is positioned over the patient's body part.

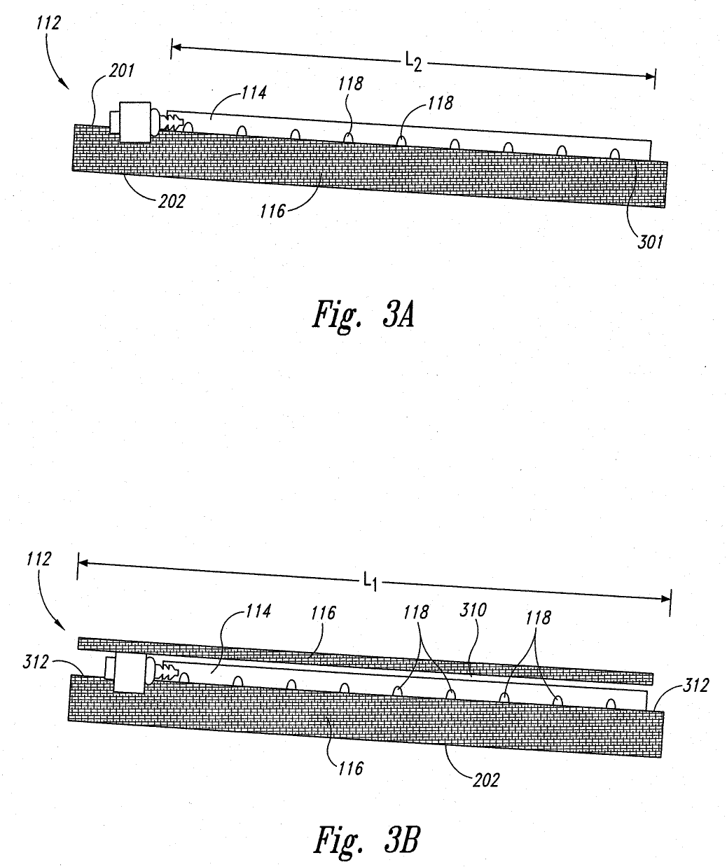

In some arrangements, the air delivery tube 114 can be coupled to the exterior surface 201 of the sleeve 112 such that the stockinette material 116 is positioned to be between the air delivery tube 114 and the patient's skin 104 (shown in FIG. 1). For example, FIG. 3A illustrates a portion of the cast ventilation sleeve 112 (indicated in FIG. 1) with the air delivery tube 114 attached to the exterior surface 201 of the stockinette material 116, and in accordance with an embodiment of the disclosure. In this arrangement, the tube 114 can be provided with the holes 118 substantially oriented toward the stockinette material 116 such that pressurized air can be dispersed through the stockinette material and toward the patient's skin 104 along the tube length L₂, and therefore, the patient's encased body part. In another arrangement, not shown, the air delivery tube 114 can be coupled to the interior surface 202 of the sleeve 112 and be in direct contact with the patient's skin 104. In either arrangement, the holes 118 can be disposed along a first side 301 of the air delivery tube 114, as illustrated in FIG. 3A.

In the illustrated embodiment the first side 301 is positioned to face the patient's skin 104. In other embodiments, the holes 118 can be positioned to face a direction other than the patient's skin 104. For example, the holes 118 may face at an angle so that air can disperse substantially parallel to the patient's skin as well as the cast ventilation sleeve 112. In other arrangements, however, the air delivery tube 114 can be perforated with holes 118 on more that one side. It will be appreciated by one of ordinary skill that the air delivery tube 114 can have any number of holes 118 and the holes 118 can be disposed in the tube 114 long any surface in a variety of patterns or arrangements.

The holes 118 can be circular holes or other shaped holes. For example, the holes 118 may be oval, or have another elongated shape for dispersing air between the patient's skin 104 and the cast 100. The holes 118 can be cut, punched, laser cut, or otherwise disposed in the air delivery tube material by processes well known in the art. In other arrangements, the holes 118 may include slits cut into the air delivery tube 114 such that air is force out of the tube along the slits when the tube is under sufficient pressure. This embodiment may allow the entire tube 114 to fill with air before air is dispersed through the holes 118.

Referring to FIGS. 1-3A together, the air delivery tube 114 can be permanently coupled to the stockinette material 116 to form the cast ventilation sleeve 112 or, in another embodiment, the tube can be removeably attached. Suitable mechanisms for attachment can include tape, mechanical fastening members, mechanical bonding agents, or chemical bonding agents. In a further embodiment, the mechanism for attachment may include sonically welding the tube 114 to a portion of or within the stockinette and/or elastomeric material 116 of the sleeve 112. In another embodiment, the tubes can be sewn to the stockinette material. It will be understood by one of ordinary skill in the art that any mechanism that allows the air delivery tube 114 to be coupled or otherwise attached to the stockinette material 116 may be used as a mechanism for attachment.

FIG. 3B is a cross-sectional view illustration of a portion of a cast ventilation sleeve 112 with an air delivery tube 114 positioned inside a sleeve receiving channel 310. In this embodiment, the sleeve 112 can include one or more receiving channels 310 formed along the sleeve length L₁. The receiving channels 310 may be formed from the stockinette material 116. For example, the sleeve 112 may be partitioned along an edge 312 by a sewn seam line, tape, adhesive, etc., to form the channel 310. In another embodiment the channel 310 can be additional flexible material that is sewn to the stockinette material 116. The receiving channel 310 can be sized to receive and releaseably (or permanently) retain the air delivery tube 114 within the channel. In one embodiment, the holes 118 can be positioned to face in the direction of the interior surface 202 and/or the patient's skin 104.

Referring to FIGS. 1 and 2 together, the first tube end 117 can be approximately coincident with the first sleeve end 203 and be positioned to receive pressurized air from the air source 120 when the air delivery nozzle 124 is engaged with both the source 120 and the tube 114. As illustrated the Figures, the air source 120 can deliver pressurized air to one or more air delivery tubes 114 via the air source line 122. In one embodiment, the air source line 122 can be provided with a manifold 126, or other mechanism to divide and control air pressure while driving the air through the integrated air delivery system 110.

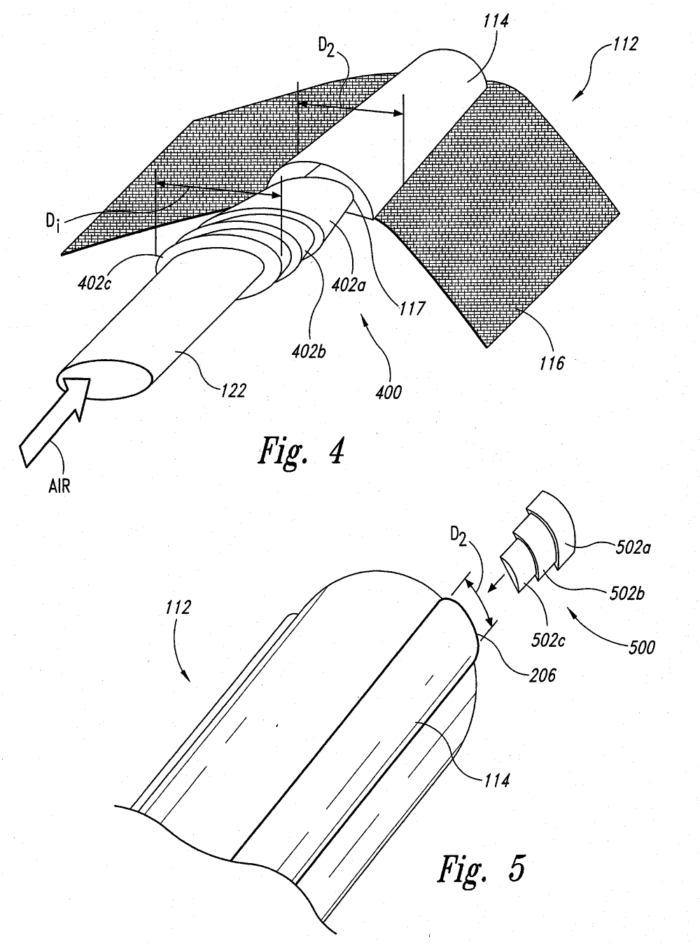

The air delivery nozzle 124 can be any fitted nozzle for adaptively engaging the tube 114 such that the pressurized air flows into the tube. For example, FIG. 4 is a perspective view of an air delivery nozzle 400 engaging an air delivery tube 114 in accordance with an embodiment of the disclosure. As illustrated, the nozzle 400 can have a plurality of exteriorly formed barbs 402a-c (referred together by reference number 402) with each barb having flanges with progressively increasing cross-sectional dimensions Dᵢ. The barbs 402 can be formed as part of the air supply line 122, or the barbs 402 can be adapted to positively engage the air supply line 122 such that air directly flows through the line and past the series of barbs 402. The nozzle 400 can be inserted into the first tube end 117 until one of the barbs 402a-c provides a suitable cross-sectional dimension to match a tube cross-sectional dimension D₂ such that the first tube end is positively sealed by insertion of the nozzle 400. The nozzle 400 can be formed from any suitable material for molding barbs (e.g., plastic, metal, ceramics, etc.). In one embodiment, the nozzle 400 and/or barbs 402 can be formed from rubber, silicone, or other semi-pliable material for forming a seal between the barbs 402 and the tube 114. Because the nozzle 400 can have a plurality of barbs 402 with increasing cross-sectional dimensions Dᵢ, the nozzle 400 can be a universal nozzle that allows the air supply 120 (shown in FIG. 2) to support air delivery to air delivery tubes 114 having a variety of tube cross-sectional dimensions D₂. In other embodiments, the nozzle 400 can have other configurations, such as engaging the tube 114 to envelope an outer surface of the tube for administering pressurized air delivery.

Referring back to FIG. 2, and in some embodiments, the second tube end 206 can remain open to air flow, such that air may flow along the air delivery tube 114 and leave the integrated air delivery system 110 via the second tube end. However, in other embodiments, the system 110 can be provided with a closure mechanism for sealing the second tube end 206 and preventing the pressurized air from escaping the system 110 via that identified route. As illustrated in FIG. 2, the system 110 can be provided with closure plugs 208 sized either to fit into or around the second tube end 206 to block potentially escaping air.

FIG. 5 is a perspective view of an embodiment of an air delivery tube closure plug 500 in accordance with the disclosure. The closure plug 500 can be shaped to seal air delivery tubes 114 having a variety of tube cross-sectional dimensions D₂. For example, the plug 500 can have barbs 502a-c or otherwise step wise increases in size to fit within with the tube 114 for sealing the tube. In other embodiments, however, other closure mechanisms can be used to effectively seal the second tube end 206 such as a piece of tape (not shown) ensuring that the second tube end 206 is in a closed confirmation.

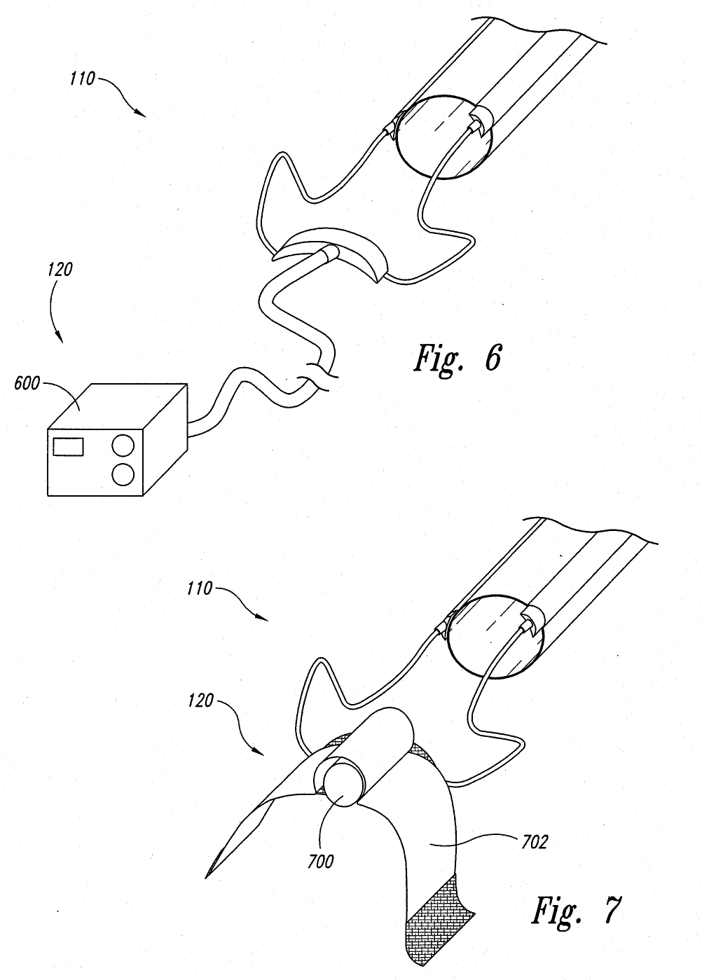

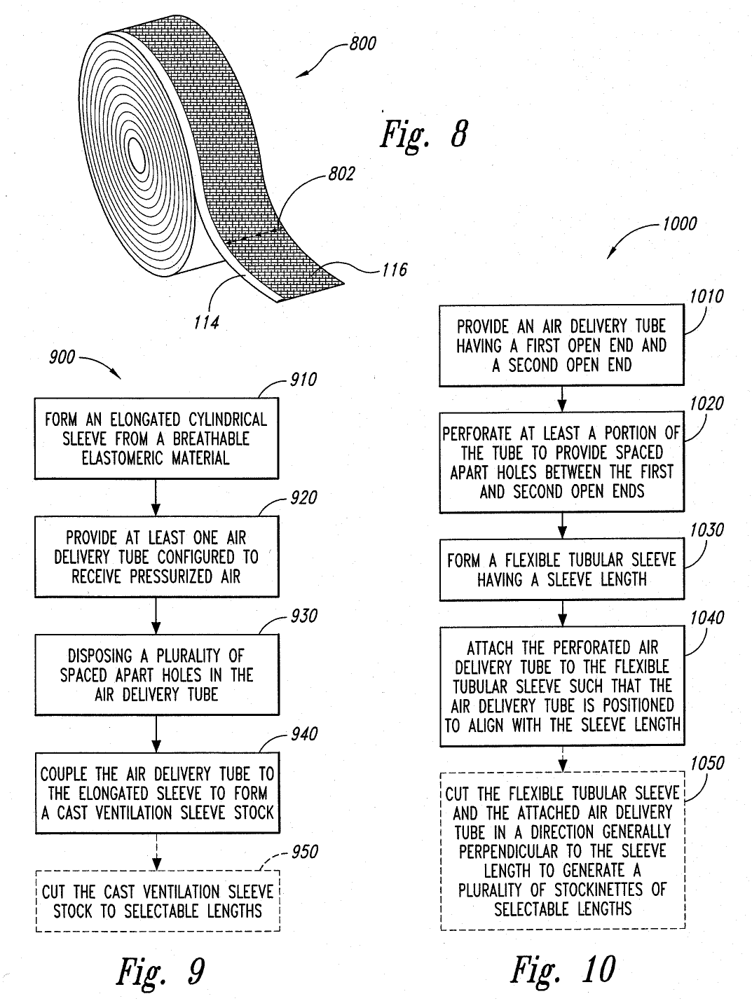

FIGS. 6 and 7 are illustrations of air supplies 120 that can be used to supply pressurized air to the integrated air delivery system 110. In one embodiment, illustrated in FIG. 6, the air source 120 can be a portable or semi-portable fan system 600. In another embodiment, illustrated in FIG. 7, the air source 120 can be a pressurized can of air 700. As illustrated in FIG. 7, the can or air can be carried by a patient via a harness 702 or other transportable attachment device. In other embodiments, not shown, the air source 120 can be a pressurized air tank or the like.

While the air source 120 can contain and provide pressurized natural air, the air source may also contain any other gas mixture suitable for the air delivery system 110 for providing relief from symptoms associated with wearing orthopedic casts 100 (e.g., gasses containing oxygen, nitrogen, hydrogen, etc.). Additionally, the air and/or other gas supply 120 can be configured to contain medicinal reagents mixed within for administration to the patient's skin 104 beneath the cast 100. For example, antimicrobial reagents, oils, humectants, and other medicinal compositions suitable for skin can be premixed with the air/gas supply, or in another embodiment, the medicinal composition can be mixed in the supply lines 122 just prior to delivery to the system 110.

Typically, medical practitioners purchase and/or have stocks of the reagents necessary to mold and create an orthopedic cast for a patient. Many of these supplies are purchased in bulk and the materials are used in only portions necessary for making a cast sized for the particular injury. For example, medical practitioners forming a conventional cast will first set the broken bone, if necessary, clean the skin in the inured region and take measurement of the size/type of cast needed. Stockinette material can be cut to size from a roll of material, placed over the patient's injured region, and the cast can be formed over the stockinette. In some applications, ventilation devices can be applied to conventional cast systems by addition of air tubes, additional wrapped layers and or venting systems formed through the cast while the cast is hardening. These systems are time consuming and require additional materials, thereby increasing he overall cost and complexity of the cast. Moreover, the medical practitioner must practice and perfect his/her skills prior to applying or building these ventilation systems into the cast.

In contrast to the conventional systems, the air delivery system 110 described herein provides a simple process for applying air delivery without introducing additional steps or layers to the formation of the cast. The cast ventilation sleeve 112 of the system 110 can be less expensive to manufacture and, therefore, have benefits beyond time saved when treating a patient. In one example, the cast ventilation sleeve 112 can be manufactured and provided as an elongated sleeve.

FIG. 8 illustrates a roll 800 of cast ventilation sleeve stock in accordance with an embodiment of the disclosure. The roll 800 can have a roll length (not shown), wherein the roll length, or elongated sleeve length, is greater than an individual cast ventilation sleeve length L₁ (or any length suitable for forming a single air delivery system 110 beneath an orthopedic cast 100). For example, the roll 800 may have a roll length of 75 feet. The roll 800 can have a similar look and feel to the conventional rolls of stockinette material typically used by medical practitioners. Moreover, medical practitioners can cut the roll 800 through the stockinette material 116 and the air delivery tubes 114 in a direction 802 generally perpendicular to the roll length to provide a cast ventilation sleeve 112 (as shown in FIG. 2) with a selectable sleeve length L₁ that can be used beneath a cast 100. A single roll 800 can, therefore, provide a plurality of cast ventilation sleeves 112 cut to any selectable size and applied to the patient beneath a cast using the same cast forming procedure as conventional casts not having any type of ventilation device.

Another aspect of the present disclosure is directed to methods of manufacturing cast ventilation sleeves and stockinettes for ventilating orthopedic casts. FIG. 9 is a flow chart of an embodiment of a method 900 for manufacturing cast ventilation sleeves. The method 900 can include forming an elongated cylindrical sleeve from a breathable elastomeric material (block 910). The elongated sleeve can have a sleeve length with a first sleeve end and a second sleeve end opposite from the first sleeve end, and have a first major cross-sectional dimension. The elastomeric material can be configured to expand to a second major cross-sectional dimension greater than the first major cross-sectional dimension. The method 900 can further include providing at least one air delivery tube configured to receive pressurized air (block 920). The air delivery tube can have a tube length with a first tube end and a second tube end opposite from the first tube end. The method 900 can also include disposing a plurality of spaced apart holes in the air delivery tube (block 930). The holes can be positioned to disperse the pressurized air along the tube length. Additionally the method 900 can include coupling the air delivery tube to the elongated sleeve to form a cast ventilation sleeve stock (block 940). The air delivery tube can be positioned to be generally parallel to the sleeve length such that the first tube end is oriented toward the first sleeve end and the second tube end is oriented toward the second sleeve end. Optionally, the method 900 can include cutting the cast ventilation sleeve stock to selectable lengths to form a plurality of individual cast ventilation sleeves configured for use beneath orthopedic casts (block 950).

FIG. 10 is a flow chart of an embodiment of a method 1000 for manufacturing stockinettes for ventilating orthopedic casts. The method 1000 can include providing an air delivery tube having a first open end and a second open end (block 1010). After providing the air delivery tube, the method 1000 can include perforating at least a portion of the air delivery tube to provide spaced apart holes between the first and second open ends (block 1020). The method 1000 can further include forming a flexible tubular sleeve having a sleeve length (block 1030). The flexible tubular sleeve can be formed from a material suitable to be worn next to a patient's skin. The method 1000 can also include attaching the perforated air delivery tube to the flexible tubular sleeve such that the air delivery tube is positioned to align with the sleeve length (block 1040). Optionally, the method 1000 can include cutting the flexible tubular sleeve and the attached air delivery tube in a direction generally perpendicular to the sleeve length to generate a plurality of stockinettes of selectable lengths (block 1050).

From the foregoing, it will be appreciated that specific embodiments of the disclosure have been described herein for purposes of illustration, but that various modifications may be made without deviating from the disclosure. Furthermore, aspects of the disclosure described in the context of particular embodiments may be combined or eliminated in other embodiments. Further, while features and characteristics associated with certain embodiments of the disclosure have been described in the context of those embodiments, other embodiments may also exhibit such features and characteristics, and not all embodiments need necessarily exhibit such features and characteristics to fall within the scope of the disclosure. Accordingly, the disclosure is not limited, except as by the appended claims.

I/We claim:

1. A method of manufacturing cast ventilation sleeves, the method comprising:
forming an elongated cylindrical sleeve from a breathable elastomeric material, the elongated sleeve having a sleeve length with a first sleeve end and a second sleeve end opposite from the first sleeve end and having a first major cross-sectional dimension, and wherein the elastomeric material is configured to expand to a second major cross-sectional dimension greater than the first major cross-sectional dimension;
providing at least one air delivery tube configured to receive pressurized air, the air delivery tube having a tube length with a first tube end and a second tube end opposite from the first tube end;
disposing a plurality of spaced apart holes in the air delivery tube, the holes positioned to disperse the pressurized air along the tube length; and
coupling the air delivery tube to the elongated sleeve to form a cast ventilation sleeve stock, wherein the air delivery tube is positioned to be generally parallel to the sleeve length such that the first tube end is oriented toward the first sleeve end and the second tube end is oriented toward the second sleeve end.

2. The method of claim 1 wherein the pressurized air is provided by a releaseably engaged air supply, and wherein the air supply is configured to dispense pressurized air via one or more air supply lines.

3. The method of claim 1 wherein the first sleeve end is coincident with the first tube end and the second sleeve end is coincident with the second tube end.

4. The method of claim 1 wherein coupling the air delivery tube to the elongated sleeve includes coupling a plurality of air delivery tubes, the air delivery tubes being spaced apart from other ones of the air delivery tubes.

5. The method of claim 4 wherein each of the air delivery tubes is moveable with a portion of the elongated sleeve relative to the other ones of the air delivery tubes.

6. The method of claim 1 wherein coupling the air delivery tube includes adhesively attaching the air delivery tube to the elongated sleeve.

7. The method of claim 1 wherein coupling the air delivery tube includes sewing the air delivery tube to the elongated sleeve.

8. The method of claim 1 wherein the elongated sleeve includes one or more receiving channels formed along the sleeve length, the receiving channel configured to receive and releaseably retain the air delivery tube, and wherein coupling the air delivery tube includes inserting the air delivery tube into the receiving channel.

9. The method of claim 1 wherein disposing a plurality of spaced apart holes in the air delivery tube includes disposing holes such that the holes are aligned along a first side of the tube, and wherein coupling the air delivery tube includes coupling the air delivery tube to the elongated sleeve such that the holes are facing an interior portion of the sleeve.

10. The method of claim 1 wherein the elongated sleeve and the air delivery tube is configured to be cut to selectable lengths smaller than the sleeve length, and wherein the selectable length is selected based upon a desired length of an orthopedic cast.

11. The method of claim 1 wherein after coupling the air delivery tube to the elongated sleeve to form the cast ventilation sleeve stock, the method further comprises cutting the cast ventilation sleeve stock to selectable lengths to form a plurality of individual cast ventilation sleeves configured for use beneath orthopedic casts.

12. A method of manufacturing stockinettes for ventilating orthopedic casts, the method comprising:
providing an air delivery tube having a first open end and a second open end;
perforating at least a portion of the air delivery tube to provide spaced apart holes between the first and second open ends;
forming a flexible tubular sleeve having a sleeve length, and wherein the flexible tubular sleeve is formed from a material suitable to be worn next to a patient's skin; and
attaching the perforated air delivery tube to the flexible tubular sleeve such that the air delivery tube is positioned to align with the sleeve length.

13. The method of claim 12 wherein the air delivery tube is configured to receive pressurized air and disperse the pressurized air through the holes.

14. The method of claim 12, further comprising cutting the flexible tubular sleeve and the attached air delivery tube in a direction generally perpendicular to the sleeve length to generate a plurality of stockinettes of selectable lengths.

15. A stock of flexible material for use as a liner between a patient's skin and an orthopedic cast, the stock comprising:
an elongated sleeve of breathable material having a sleeve length and having an inner sleeve surface and an outer sleeve surface; and
an air delivery tube coupled to the elongated sleeve, the air delivery tube including a plurality of spaced apart holes along a tube length, wherein the air delivery tube is in an orientation generally parallel to the sleeve length, and wherein the air delivery tube is configured to receive pressurized air.

16. The stock of flexible material of claim 15 wherein the air delivery tube includes a plurality of air delivery tubes, the air delivery tubes being spaced apart from and adjacent other ones of the air delivery tubes.

17. The stock of flexible material of claim 16 wherein each of the air delivery tubes is moveable with a portion of the elongated sleeve relative to the other ones of the air delivery tubes.

18. The stock of flexible material of claim 15 wherein the air delivery tube is coupled to the elongated sleeve with an adhesive.

19. The stock of flexible material of claim 15 wherein the air delivery tube is sewn to the elongated sleeve.

20. The stock of flexible material of claim 15 wherein the elongated sleeve includes one or more receiving channels formed along the sleeve length, and wherein the delivery tube is inserted into the receiving channel.

21. The stock of flexible material of claim 15 wherein the air delivery tube is sonically welded onto a portion of the elongated sleeve.

22. A cast ventilation system, the system comprising:
a cast ventilation sleeve configured to be worn by a patient beneath an orthopedic cast, the cast ventilation sleeve including—
an elastomeric material configured to be worn next to a patient's skin, wherein the elastomeric material surrounds and expands to cover a patient body part; and
an air delivery tube coupled to an outer surface of the elastomeric material, the air delivery tube having a plurality of spaced apart air dispersing holes, wherein the air dispersing holes are positioned to deliver air through the elastomeric material and toward the patient's skin;
wherein the cast ventilation sleeve is formed from a manufacturing process, the process including—
(a) forming an elongated cylindrical sleeve from the elastomeric material, the elongated sleeve having a first length;
(b) coupling the air delivery tube to the elongated sleeve such that the air delivery tube extends over the first length; and
(c) cutting the elongated sleeve and the air delivery tube in a direction generally perpendicular to the first length to form the cast ventilation sleeve having a second length less than the first length, the second length selected to accommodate an orthopedic cast length; and
an air supply releaseably connected to the air delivery tube for providing pressurized air to the air delivery tube.

23. The cast ventilation system of claim 22 wherein the air delivery tube has a first tube end configured to be releaseably engaged by an air supply line and a second tube end opposite from the first tube end, and wherein the second tube end is positively engaged by a closure mechanism to prevent the pressurized air from escaping the second tube end.

24. The cast ventilation system of claim 22 wherein the pressurized air includes at least one medicinal reagent.
